(19) United States
(12) Patent Application Publication
(10) Pub. No.: US 2010/0160753 A1
(43) Pub. Date: Jun. 24, 2010

(54) WRIST BLOOD PRESSURE MONITOR WITH AN OXIMETER

(75) Inventors: Weihu Wang, Beijing (CN); Lei Chen, Beijing (CN)

Correspondence Address: SEYFARTH SHAW LLP, WORLD TRADE CENTER EAST, TWO SEAPORT LANE, SUITE 300, BOSTON, MA, 02210-2028, US

(73) Assignee: Beijing Choice Electronic Technology Co., Ltd., Beijing (CN)

(21) Appl. No.: 12/161,120

(22) PCT Filed: Aug. 27, 2007
(86) PCT No.: PCT/CN2007/070567

§ 371 Date: Jul. 16, 2008

(51) Int. Cl. A61B 5/02

(52) U.S. Cl. 600/324

(57) ABSTRACT

A wrist blood pressure monitor with an oximeter, characterized in that the wrist blood pressure monitor and the oximeter are arranged on the same base; the length direction of the wrist blood pressure monitor is parallel to the width direction of an armband; and a finger hole of the oximeter is oriented to be vertical to the width direction of the armband. The wrist blood pressure monitor with the oximeter according to the present invention can measure the blood pressure and the blood oxygen at the same time, so that the user can not only operate conveniently, but also acquire more accurate measuring results; or the blood pressure and the blood oxygen are measured independently, so that the user can operate in great convenience.

### TECHNICAL FIELD

The present invention relates to a wrist blood pressure monitor, in particular to a wrist blood pressure monitor with an oximeter.

More particularly, the present invention relates to a portable wrist blood pressure monitor which can simultaneously measure blood pressure and blood oxygen. The user can not only operates conveniently, but also acquire more accurate measuring results.

Furthermore, the present invention relates to an integrated wrist blood pressure monitor which can independently measure blood pressure and independently measure blood oxygen, so that the user operates conveniently.

### BACKGROUND OF THE INVENTION

In the prior art, the sphygmomanometer (the so-called blood pressure monitor) and the oximeter have to be used respectively in the case that the blood pressure and the blood oxygen are needed to be measured simultaneously. There exist at least two problems, i.e. firstly, operational inconvenience of changing instruments due to use of two different instruments; and secondly, intensified activity for action of human body during changing instruments, which will directly have influence on the measuring accuracy of latter instrument, and for which the measuring values of the blood pressure and the blood oxygen at the same moment cannot be obtained.

For this reason, a wrist blood pressure monitor which can simultaneously measure the blood pressure and the blood oxygen has once appeared on the market, in which the wrist blood pressure monitor and the oximeter are connected through a spiral lead, such that the blood pressure measurement on the left wrist and the blood oxygen measurement on the left finger are achieved at the same time. However, in this separated structure, the vein is compressed, so the blood oxygen cannot be accurately measured when the blood pressure is measured. In addition, installment and detachment are needed due to a plurality of parts, thus resulting in inconvenient carrying and using.

### SUMMARY OF THE INVENTION

The object of the present invention is to provide a wrist blood pressure monitor with an oximeter which can simultaneously measure blood pressure and blood oxygen. The user can not only operate conveniently, but also acquire more accurate measuring results.

Another object of the present invention is to provide a wrist blood pressure monitor with an oximeter which can independently measure blood pressure and independently measure blood oxygen, so that the user operates conveniently.

Thus, according to one aspect of the present invention, there is provided a wrist blood pressure monitor with an oximeter, characterized in that the wrist blood pressure monitor and the oximeter are arranged on the same base; the length direction of the wrist blood pressure monitor is parallel to the width direction of an armband; and a finger hole of the oximeter is oriented to be vertical to the width direction of the armband.

Preferably, the wrist blood pressure monitor and the oximeter are provided with individual display screens.

Preferably, the wrist blood pressure monitor and the oximeter are provided with a common display screen.

Preferably, the wrist blood pressure monitor and the oximeter are provided with individual starting operation buttons.

Preferably, the wrist blood pressure monitor and the oximeter are provided with a common starting operation button.

Preferably, the oximeter has a pivotable cover.

Preferably, the wrist blood pressure monitor and the oximeter are provided with a common power source.

Preferably, the wrist blood pressure monitor and the oximeter are provided with individual power sources.

Preferably, the measuring data of at least one of the wrist blood pressure monitor and the oximeter can be displayed in different oriented directions changing from one to another.

Preferably, the wrist blood pressure monitor with the oximeter has a package box which can be used as a wrist cushion during measurement.

According to the present invention, the wrist blood pressure monitor has a function of blood oxygen measurement. If the user desires to measure the blood pressure and the blood oxygen, the blood pressure and the blood oxygen can be measured simultaneously. It is unnecessary that the wrist blood pressure monitor is used first, and then the oximeter is used thereafter; or the oximeter is used first, and then the wrist blood pressure monitor is used thereafter. Therefore, the problems resulted from changing instruments no longer exist, so that the user can operate in great convenience.

Especially, because the blood oxygen on the right finger can be measured while the blood pressure on the left wrist is measured, the problem of influence on the measuring accuracy of the blood oxygen due to compression on the vein no longer exist.

Especially, because the blood pressure or the blood oxygen can be measured independently, the user can operate in great convenience.

Especially, the wrist blood pressure monitor with the oximeter according to the present invention is an integrated cordless structure without accessory. Therefore, it is convenient for carrying. It is with only a few buttons, being able to achieve one-button operation, so that it is suitable for elderly and home usage.

Especially, the wrist blood pressure monitor with the oximeter according to the present invention adopts a modular structure. A lower cover of the oximeter is fixed on the base of the wrist blood pressure monitor, and an upper cover can be upgraded, so that the display screens of different types can be changed by only changing the mould of the upper cover, and the displaying manners of different types may be adopted. The Bluetooth function and the wireless transmission manner, etc. can be added.

Especially, the wrist blood pressure monitor with the oximeter according to the present invention cancels the externally connected detector and forms an integral structure without separate parts, so that only one display screen and one public power source may be adopted.

Further, the oximeter can be mounted on the base after rotating 90 degrees along its longitudinal axis in the case that the wrist blood pressure monitor and the oximeter are provided with the common display screen. At this time, the opening and closing between an upper shell and a lower shell of the oximeter are no longer along the thickness direction of the wrist blood pressure monitor any more, but are changed to be along the longitudinal direction of the wrist blood pressure monitor. In this case, the finger pad of the right finger in which the blood oxygen is measured is not toward below any more, but toward the chest of the measured person, during the measurement, therefore, the measured person can feel more comfortable.

### BRIEF DESCRIPTION OF THE DRAWINGS

FIG. 1 is a perspective view of a wrist blood pressure monitor with an oximeter according to the present invention.

FIG. 2 is a perspective view of another embodiment of a wrist blood pressure monitor with an oximeter according to the present invention.

### DETAILED DESCRIPTION OF THE PREFERRED EMBODIMENTS

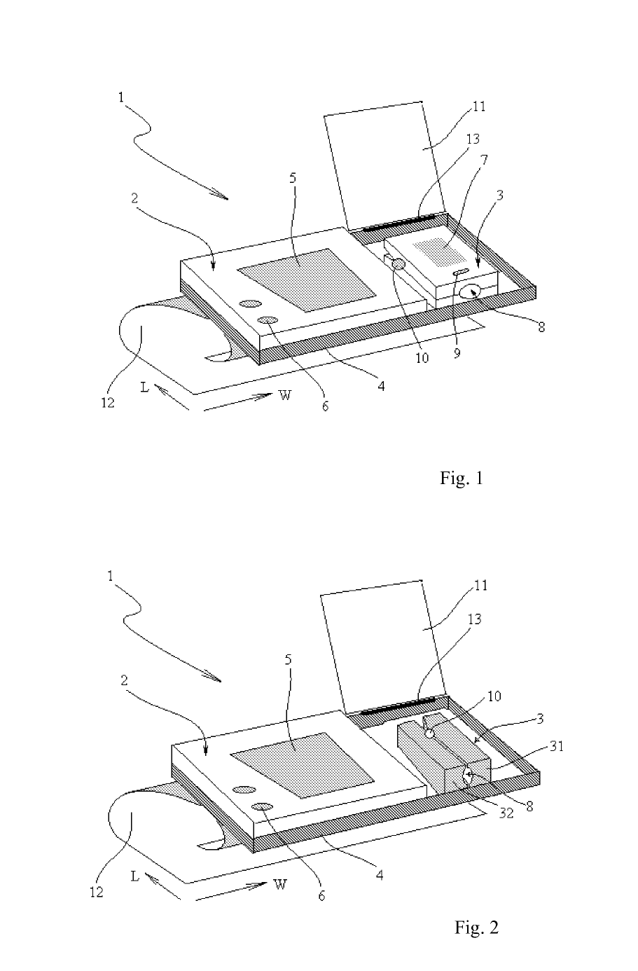

Next, an embodiment of the present invention is described in more detail in combination with FIG. 1.

In the embodiment, a wrist blood pressure monitor 1 with an oximeter according to the present invention comprises a wrist blood pressure monitor 2 and an oximeter 3. The wrist blood pressure monitor 2 and the oximeter 3 are both mounted on a base 4. The wrist blood pressure monitor 2 has a display screen 5 and an operation button 6. The oximeter 3 has a display screen 7, a finger hole 8, an operation button 9, and a pivot 10. The wrist blood pressure monitor and the oximeter are provided with a common power source, such as two common batteries.

Especially, the oximeter 3 may be provided with a cover 11 with a pivot 13.

The wrist blood pressure monitor 1 with the oximeter according to the present invention is provided with an armband 12, wherein the width direction of the armband 12 is represented by an arrow W, and the length direction is represented by an arrow L.

The length direction (not illustrated) of the wrist blood pressure monitor 1 is parallel to the width direction W of the armband, while the finger hole 8 of the oximeter is oriented to be vertical to the width direction W of the armband.

In another embodiment, the wrist blood pressure monitor 2 and the oximeter 3 are provided with a common display screen.

Further, the oximeter 3 can be mounted on the base 4 after rotating 90 degrees along its longitudinal axis compared to the first embodiment in the case that the wrist blood pressure monitor 2 and the oximeter 3 are provided with the common display screen 5. At this time, the opening and closing between an upper shell 31 and a lower shell 32 of the oximeter 3 are no longer along the thickness direction of the wrist blood pressure monitor 2 any more, but are changed to be along the longitudinal direction of the wrist blood pressure monitor 2. In this case, the finger pad of the right finger in which the blood oxygen is measured is no longer toward below any more, but is changed to face the chest of measured person, during the measurement, therefore, the measured person feels more comfortable.

In another embodiment, the wrist blood pressure monitor 2 and the oximeter 3 are provided with a common starting operation button.

At this time, the wrist blood pressure monitor 2 and the oximeter 3 can be started at the same time by only triggering one button, thus further providing convenience for users.

In another embodiment, the wrist blood pressure monitor 2 and the oximeter 3 are provided with individual power sources.

In another embodiment, the measuring data of at least one of the wrist blood pressure monitor 2 and the oximeter 3 can be displayed in different oriented directions changing from one to another.

In another embodiment, the wrist blood pressure monitor 2 with the oximeter 3 has a package box which can be used as a wrist cushion during measurement.

The present invention is described above in detail according to a plurality of embodiments. However, those skilled in the art should understand that various modifications and improvements can be made on the present invention without departing from the spirit of the present invention, and the scope of protection is defined by the attached claims of the present invention.

1. A wrist blood pressure monitor with an oximeter, characterized in that the wrist blood pressure monitor and the oximeter are arranged on the same base; the length direction of the wrist blood pressure monitor is parallel to the width direction of an armband; and a finger hole of the oximeter is oriented to be vertical to the width direction of the armband.

2. The wrist blood pressure monitor with the oximeter according to claim 1, characterized in that the Bluetooth is provided; and the measuring data are transmitted by wireless transmission manner.

3. The wrist blood pressure monitor with the oximeter according to claim 1, characterized in that the wrist blood pressure monitor and the oximeter are provided with a common display screen.

4. The wrist blood pressure monitor with the oximeter according to claim 1, characterized in that the wrist blood pressure monitor and the oximeter are provided with individual starting operation buttons.

5. The wrist blood pressure monitor with the oximeter according to claim 1, characterized in that the wrist blood pressure monitor and the oximeter are provided with a common starting operation button.

6. The wrist blood pressure monitor with the oximeter according to claim 1, characterized in that the oximeter has a pivotable cover.

7. The wrist blood pressure monitor with the oximeter according to claim 1, characterized in that the wrist blood pressure monitor and the oximeter are provided with a public power source.

8. The wrist blood pressure monitor with the oximeter according to claim 1, characterized in that the wrist blood pressure monitor and the oximeter are provided with individual power sources.

9. The wrist blood pressure monitor with the oximeter according to claim 1, characterized in that the measuring data of at least one of the wrist blood pressure monitor and the oximeter can be displayed in different oriented directions changing from one to another.

10. The wrist blood pressure monitor with the oximeter according to claim 1, characterized in that the wrist blood pressure monitor with the oximeter has a package box which can be used as a wrist cushion during measurement.
